(19) United States
(12) Patent Application Publication
(10) Pub. No.: US 2024/0248073 A1
(43) Pub. Date: Jul. 25, 2024

(54) METHOD AND EQUIPMENT FOR DETERMINING AN ESTIMATED LIFE AND REMAINING USEFUL LIFE OF A MECHANICAL DEVICE

(71) Applicant: Aktiebolaget SKF, Göteborg (SE)

(72) Inventors: Weiqi GUO, Shanghai (CN); Wenjun LI, Shanghai (CN); Gang CHENG, Shanghai (CN); Xin LIU, Beijing (CN); Bin ZHANG, Beijing (CN)

(21) Appl. No.: 18/409,888

(22) Filed: Jan. 11, 2024

(30) Foreign Application Priority Data
Jan. 20, 2023 (CN) 202310083371.X

(51) Int. Cl. G01N 33/28; F03D 17/00

(52) U.S. Cl. CPC G01N 33/2888; G01N 33/2858; F03D 17/011

(57) ABSTRACT

A method for determining an estimated life of a mechanical device includes (1) acquiring a load spectrum of the mechanical device within a predetermined time range; (2) acquiring a lubricant state of the mechanical device within the predetermined time range; (3) determining a plurality of operating states experienced by the mechanical device within the predetermined time range based on the acquired load spectrum and the acquired lubricant state, and (4) determining an estimated life of the mechanical device based on the plurality of operating states of the mechanical device within the predetermined time range. Each operating state corresponds to a particular range of load values in the load spectrum and a particular degree of contamination in the lubricant state. The method may also determine the remaining useful life of a mechanical device. The method may be implemented in a device for determining the estimated life of a mechanical device.

### CROSS-REFERENCE TO RELATED APPLICATIONS

This application claims priority to Chinese Application No. 202310083371.X, filed Jan. 20, 2023, the entirety of which is hereby incorporated by reference.

### FIELD

The present disclosure relates to a method for determining an estimated life of a mechanical device, a method for determining a remaining useful life of a mechanical device, and an equipment for determining an estimated life of a mechanical device.

### BACKGROUND

Mechanical devices have rated life according to their design and manufacturing standards, and the remaining useful life of the mechanical devices decreases as it is put into operation. Depending on the length of the remaining useful life of mechanical device, maintenance personnel can target the maintenance of mechanical device, prepare it for replacement or repair, or optimize its operational efficiency and avoid unplanned downtime.

However, in the long-term operation of mechanical device, such as wind turbines, the loads, rotational speeds, ambient temperatures, corrosion, and lubricant state of the mechanical device will constantly change, and these changing factors will affect the rated life of the mechanical device, making the rated life no longer constant. In this case, the traditional method of predicting the remaining useful life of mechanical device is not effective.

### SUMMARY

The present disclosure presents a method for determining an estimated life of a mechanical device and a method for determining a remaining useful life of a mechanical device. The method according to the present disclosure takes into account load variations in the operation of a mechanical device as well as changes in the lubricant state, thereby real-time and accurate determination of the life of the mechanical device and the remaining useful life can be achieved.

The present disclosure provides a method for determining an estimated life of a mechanical device. The method includes acquiring a load spectrum of the mechanical device within a predetermined time range; acquiring a lubricant state of the mechanical device within the predetermined time range; determining a plurality of operating states experienced by the mechanical device within the predetermined time range based on the acquired load spectrum and the acquired lubricant state, wherein each operating state corresponds to a particular range of load values in the load spectrum and a particular degree of contamination in the lubricant state; and determining an estimated life of the mechanical device based on the plurality of operating states of the mechanical device within the predetermined time range.

In an embodiment according to the present disclosure, determining an estimated life of the mechanical device based on the plurality of operating states of the mechanical device within the predetermined time range includes: Determining the corresponding reference life of the mechanical device for each of the plurality of operating states, and determining an estimated life of the mechanical device based on the reference life corresponding to each of the plurality of operating states.

In an embodiment according to the present disclosure, Determining the corresponding reference life of the mechanical device for each of the plurality of operating states includes: Determining the corresponding reference life of the mechanical device for each of the plurality of operating states according to a predetermined criterion or methodology regarding the life of the mechanical device.

In an embodiment according to the present disclosure, determining a plurality of operating states experienced by the mechanical device within the predetermined time range includes: determining an operating time for each of the plurality of operating states; wherein determining an estimated life of the mechanical device based on the reference life corresponding to each of the plurality of operating states includes: using the proportion of the operating time of each operating state to the predetermined time range as a weighted weight, and calculating a weighted mean of the respective reference life as the estimated life.

In an embodiment according to the present disclosure, the weighted mean includes a weighted harmonic mean, a weighted arithmetic mean, and a weighted geometric mean.

In an embodiment according to the present disclosure, the load spectrum of the mechanical device includes at least one of the following: a temperature spectrum, a torque spectrum, a rotational speed spectrum, a radial force spectrum and an axial force spectrum of the mechanical device.

In an embodiment according to the present disclosure, the method further includes preprocessing the load spectrum, the preprocessing including at least one of the following: removing anomalous load values from the load spectrum and replacing null values in the load values by interpolation.

In an embodiment according to the present disclosure, acquiring a lubricant state of the mechanical device within the predetermined time range includes: acquiring a discrete lubricant state of the mechanical device within the predetermined time range and deriving a continuous lubricant state within the predetermined time range based on the discrete lubricant state and a time point at which the lubricant is cleaned within the predetermined time range.

In an embodiment according to the present disclosure, the particular degree of contamination includes: lightly contaminated, moderately contaminated and heavily contaminated.

In an embodiment according to the present disclosure, the method further includes predetermining a maximum range of load values, the maximum range of load values being divided into a plurality of sub-ranges, wherein the particular range of load values includes at least one of the sub-ranges.

Embodiments of the present disclosure also provide a method for determining a remaining useful life of a mechanical device. The method includes determining an estimated life of the mechanical device by using the method for determining an estimated life of a mechanical device mentioned above, and determining the time the mechanical device has been in use, and determining the remaining useful life of the mechanical device based on the estimated life of the mechanical device and the time the mechanical device has been in use.

Embodiments of the present disclosure also provide an equipment for determining an estimated life of a mechanical device, the equipment includes a first acquisition unit for acquiring a load spectrum of the mechanical device within a predetermined time range; a second acquisition unit for acquiring a lubricant state of the mechanical device within the predetermined time range; an operating state determination unit that is connected to the first acquisition unit and the second acquisition unit and acquires the load spectrum and the lubricant state and, determines a plurality of operating states experienced by the mechanical device within the predetermined time range based on the load spectrum and the lubricant state, wherein each operating state corresponds to a particular range of load values in the load spectrum and a particular degree of contamination; an estimated life determination unit that is connected to the operating state determination unit, determines an estimated life of the mechanical device based on the plurality of operating states of the mechanical device within the predetermined time range.

In determining the estimated life or remaining useful life of the mechanical device, the method for determining the estimated life of the mechanical device and the method for determining the remaining useful life of the mechanical device according to the present disclosure can take into account all the operating states, i.e., combinations of different load conditions and different lubricant states, that the mechanical device has experienced from the time it was put into operation until the current time point. Since different operating states have different effects on the life of the mechanical device, the methods according to the present disclosure are capable of accurately determining or adjusting the estimated life of the mechanical device based on the various operating states experienced by the mechanical device. In addition, the number and duration of the various operating states experienced by the mechanical device can change over time. The methods according to the present disclosure can take such variations into account and are therefore can determine the life and remaining useful life of the mechanical device in real time.

### BRIEF DESCRIPTION OF THE DRAWINGS

In order to more clearly illustrate the technical solutions of the embodiments of the present disclosure, the accompanying drawings to be used in the description of the embodiments will be briefly described below. Obviously, the accompanying drawings in the following description are only some exemplary embodiments of the present disclosure, and for a person of ordinary skill in the art, other accompanying drawings can be acquired based on these drawings without creative labor.

FIG. 1 illustrates a flowchart of a method for determining an estimated life of a mechanical device according to an embodiment of the present disclosure,

FIG. 2 illustrates a load spectrum of a mechanical device according to an embodiment of the present disclosure,

FIG. 3 illustrates a lubricant state in a mechanical device according to an embodiment of the present disclosure,

FIG. 4 illustrates a lubricant state in a mechanical device according to another embodiment of the present disclosure,

FIG. 5 illustrates a flowchart of a method for determining an estimated life of a mechanical device according to another embodiment of the present disclosure,

FIG. 6 illustrates a flowchart of a method for determining an estimated life of a mechanical device according to another embodiment of the present disclosure, and

FIG. 7 illustrates an equipment for determining an estimated life of a mechanical device according to an embodiment of the present disclosure.

### DETAILED DESCRIPTION

In order to make the objects, technical solutions and advantages of the present disclosure more apparent, example embodiments according to the present disclosure will be described in detail below regarding the accompanying drawings. Obviously, the described embodiments are only a part of the embodiments of the present disclosure and not all of the embodiments of the present disclosure, and it should be understood that the present disclosure is not limited by the example embodiments described herein.

In this specification and the accompanying drawings, substantially the same or similar method steps and elements are represented by the same or similar drawing symbols, and repetitive descriptions of these method steps and elements will be omitted. Also, in the description of the present disclosure, the terms “first”, “second”, and the like are used only to differentiate descriptions and are not to be understood as indicating or implying relative importance or ordering. In embodiments of the present disclosure, unless expressly stated otherwise, “connecting” does not mean that there must be a “direct connection” or “direct contact”, but only an electrical connection is required.

In this specification and the accompanying drawings, elements are described in singular or plural form, depending on the embodiment. However, the singular and plural forms have been appropriately selected for use in the presented embodiments solely for ease of interpretation and are not intended to limit the present disclosure. Thus, the singular form may include the plural form, and the plural form may include the singular form unless the context indicates otherwise.

FIG. 1 illustrates a flowchart of a method 100 for determining an estimated life of a mechanical device In an embodiment according to the present disclosure. The method 100 includes step S110: acquiring a load spectrum of the mechanical device within a predetermined time range; step S120: acquiring a lubricant state of the mechanical device within the predetermined time range; step S130: determining a plurality of operating states experienced by the mechanical device within the predetermined time range based on the acquired load spectrum and the acquired lubricant state, wherein each operating state corresponds to a particular range of load values in the load spectrum and a particular degree of contamination in the lubricant state; and step S140: determining an estimated life of the mechanical device based on the plurality of operational states of the mechanical device within the predetermined time range.

The load spectrum describes the change in load values of the mechanical device over time. The load spectrum may be taken continuously at predetermined time intervals. The load of the mechanical device may be any variable that affects the life of the mechanical device. In an embodiment according to the present disclosure, the load spectrum of the mechanical device may e.g. include at least one of the following: a temperature spectrum, a torque spectrum, a rotational speed spectrum, a radial force spectrum, and an axial force spectrum of the mechanical device. The enumerated load spectra are only exemplary and other existing or possible future load spectra should be included herein by reference.

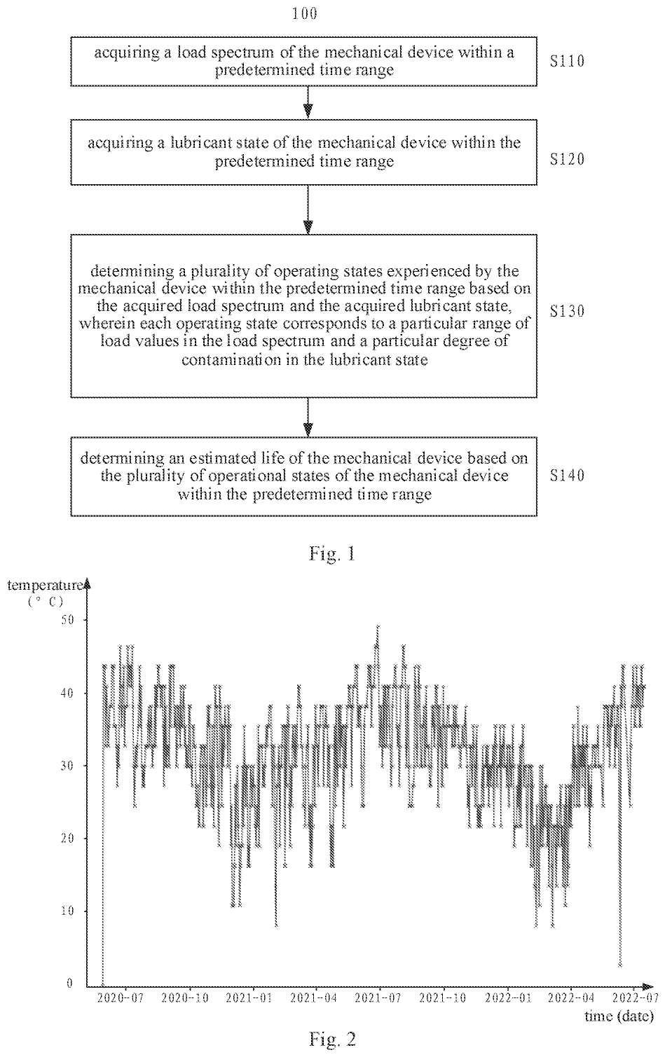

FIG. 2 illustrates a load spectrum of a mechanical device according to an embodiment of the present disclosure. In FIG. 2, temperature is illustrated exemplarily as a load, and thus FIG. 2 illustrates a temperature spectrum, i.e., temperature over time of the mechanical device. As can be seen in FIG. 2, the horizontal axis is time (date) and the vertical axis is temperature (° C.). FIG. 2 depicts the temperature variation of a mechanical device from June 2020 to June 2022, during which time the temperature of the mechanical device fluctuates between 0° C. and 50° C. The temperature of the mechanical device is continuously collected at intervals of, e.g., days or weeks. Collection intervals can also be set more intensively, e.g. hours, minutes or seconds. The load spectrum of the mechanical device may e.g. be collected by means of sensors arranged inside the mechanical device, e.g. a temperature spectrum may be collected by means of a temperature sensor, a torque spectrum by means of a torque sensor, a rotational speed spectrum by means of a rotational speed sensor, as well as a radial force spectrum and/or an axial force spectrum by means of a force sensor. The collected load spectrum may have too large or too small outliers or null values. In an embodiment according to the present disclosure, the load spectrum may e.g. be preprocessed. The preprocessing includes removing anomalous load values from the load spectrum and replacing null values in the load values by interpolation. The enumerated preprocessing of the load spectrum is only exemplary, and other existing or possible future preprocessing of the load spectrum should also be included herein by reference.

A lubricant is a substance that acts as a lubricant in mechanical device, such as lubricating oil, grease, and fluid. The lubricant state describes the change in the properties of the lubricant itself over time. Therefore, the degree of contamination of the lubricant state should be broadly understood as the degree of change in the properties of the lubricant itself. E.g., the lubricant state may be described by a content of solid particulate matter in the lubricant, e.g. in ppm (parts per million). The solid particulate matter may, e.g., be metallic particulate matter such as iron particles or maybe dust. In this regard it is possible, e.g., to sample the lubricant and determine the solid particulate matter content of the sample, and then to determine the solid particulate matter content (ppm) in the lubricant accordingly. With respect to the solid particulate matter content in the lubricant, the degree of contamination of the lubricant state indicates the level of solid particulate matter content in the lubricant. Alternatively, the lubricant state may e.g. also be characterized by a lubricant viscosity. With respect to the lubricant viscosity, the degree of contamination of the lubricant state indicates the magnitude of the lubricant viscosity.

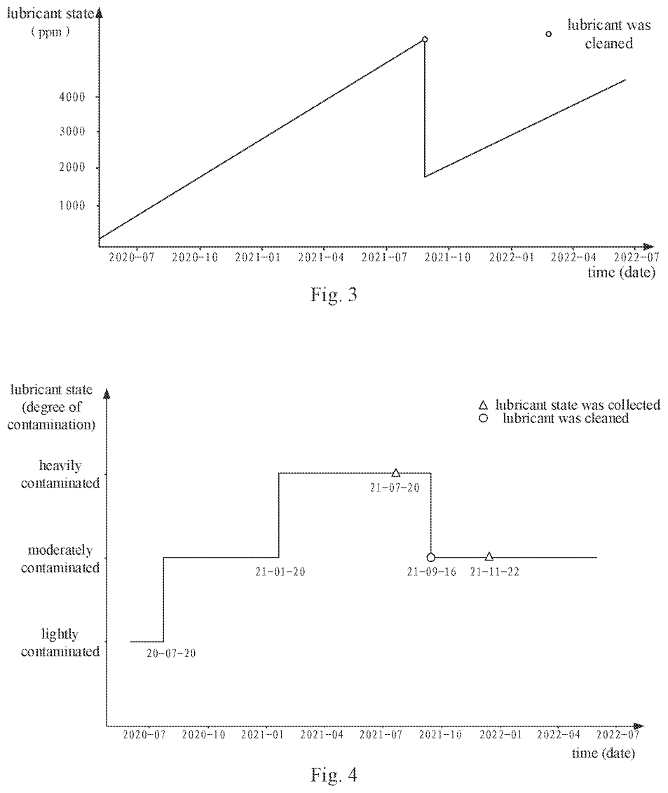

FIG. 3 illustrates a lubricant state in a mechanical device according to an embodiment of the present disclosure. In FIG. 3, the horizontal axis is time (date), the vertical axis is the lubricant state in the mechanical device in ppm, and ppm describes the amount of solid particulate matter in the lubricant. FIG. 3 illustrates, exemplarily, that from the time point of July 2020, the content of solid particulate matter in the lubricant of the mechanical device grows as the operating time of the mechanical device grows, i.e., the degree of contamination of the lubricant grows. In September 2021, the lubricant in the mechanical device was cleaned, so that the content of solid particulate matter in the lubricant decreased, and the degree of contamination of the lubricant decreased as a result. Subsequently, the content of solid particulate matter in the lubricant of the mechanical device again keeps increasing as the operating time of the mechanical device increases. The lubricant state in the mechanical device may e.g. be continuously collected at predetermined time intervals by sensors arranged in the mechanical device. Preferably, the lubricant state may e.g. be acquired at the same time interval as when the load spectrum is acquired.

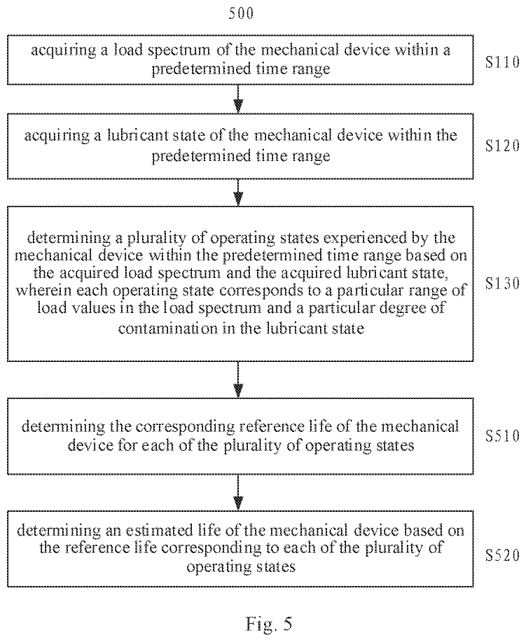

FIG. 4 illustrates a lubricant state in a mechanical device according to another embodiment of the present disclosure. Unlike describing the lubricant state of a mechanical device in ppm, in the example shown in FIG. 4, the lubricant state, i.e., the degree of contamination, is categorized as lightly contaminated, moderately contaminated, and heavily contaminated. For a lightly contaminated lubricant, the solid particulate matter content is about a few hundred ppm, however, for a heavily contaminated lubricant, the solid particulate matter content may be in the thousands of ppm. Thus, e.g., a first threshold may be set, such as 800, 1000, or 1200 ppm, below which the lubricant state is considered to be lightly contaminated; and a second threshold may be set, such as 4000 ppm, above which the lubricant state is considered to be heavily contaminated, and if it is greater than the first threshold and less than the second threshold, the lubricant state is considered to be moderately contaminated. Of course, it is also possible to classify the lubricant state into a plurality of classes in other ways, e.g., the lubricant state can be classified into 4 or 5 classes, etc. Furthermore, unlike in FIG. 3 where the lubricant state is collected continuously, in FIG. 4, the collection of the lubricant state is performed only on Jul. 20, 2021, and Nov. 22, 2021. This acquisition of the lubricant state may, e.g., be carried out in the form of an inspection report, i.e. the lubricant was sampled only on Jul. 20, 2021, and Nov. 22, 2021, and an inspection report was generated. In addition, on Sep. 16, 2021, the lubricant was cleaned.

In an embodiment according to the present disclosure, acquiring a lubricant state of the mechanical device within the predetermined time range may include: acquiring a discrete lubricant state of the mechanical device within the predetermined time range and deriving a continuous lubricant state within the predetermined time range based on the discrete lubricant state and a time point at which the lubricant was cleaned within the predetermined time range. It is known that the degree of contamination of a lubricant deteriorates with increasing time of use, so that it may be provided, e.g., that a lightly contaminated lubricant becomes moderately contaminated after a predetermined first time of use, and a moderately contaminated lubricant becomes heavily contaminated after a predetermined second time of use. The first and second use times may be different or the same. In FIG. 4, the first and second use times are exemplarily predetermined to be 6 months. It is also provided that the reverse derivation applies, i.e. that the heavily contaminated lubricant is moderately contaminated until the predetermined second use time, and that the moderately contaminated lubricant is lightly contaminated until the predetermined first use time. In addition, it is provided that the lubricant may be cleaned to change from heavily contaminated to moderately contaminated, or from moderately contaminated to lightly contaminated, or may also change from heavily contaminated to lightly contaminated. Based on the above provisions, the lubricant state over the entire predetermined time range can be deduced from the state of the collected lubricant and the time point when the lubricant was cleaned as shown in FIG. 4, i.e., the lubricant was collected as heavily contaminated on Jul. 20, 2021, and it is deduced that the lubricant has been heavily contaminated for 6 months prior to that date; the lubricant was moderately contaminated on Jan. 20, 2021 and it is deduced that the lubricant has been moderately contaminated for 6 months prior to that date; the lubricant was lightly contaminated on Jul. 20, 2020, it is deduced that the lubricant has been lightly contaminated prior to that date (until the time point when the mechanical device is activated or the lubricant is cleaned or replaced). The lubricant remained heavily contaminated from Jul. 20, 2021 until the lubricant was cleaned on Sep. 16, 2021, at which point the lubricant changed from heavily contaminated to moderately contaminated. The moderate contamination of the lubricant changed to heavy contamination (not shown) after 6 months. Alternatively, the moderate contamination may begin with the moderate contamination being collected on Nov. 22, 2021, and change to heavy contamination after 6 months (not shown).

Based on the acquired load spectrum and the acquired lubricant state it is possible to determine a plurality of operating states experienced by the mechanical device within the predetermined time range. Each operational state corresponds to a particular range of load values in the load spectrum and a particular degree of contamination in the lubricant state, i.e. the mechanical device operates at a particular range of load values and a particular degree of contamination. E.g., the temperatures)(° C. shown in FIG. 2 may e.g. be classified as [0, 10° C.], [11, 20° C.], [21, 30° C.], [31, 40° C.] and [41, 50° C.]; the lubricant states shown in FIG. 4 are classified as lightly contaminated, moderately contaminated and heavily contaminated, the plurality of operating states are, e.g., combinations of the above mentioned temperature ranges and lubricant states, i.e.

| | [0, 10° C.] | [11, 20° C.] | [21, 30° C.] | [31, 40° C.] | [41, 50° C.] |
|---|---|---|---|---|---|
| lightly | Operational | Operational | Operational | Operational | Operational |
| contaminated | state 1 | state 2 | state 3 | state 4 | state 5 |
| moderately | Operational | Operational | Operational | Operational | Operational |
| contaminated | state 6 | state 7 | status 8 | status 9 | status 10 |
| heavily | Operational | Operational | Operational | Operational | Operational |
| contaminated | status 11 | status 12 | status 13 | status 14 | status 15 |

The mechanical device may have experienced all of the operational states within the predetermined time range or may have experienced only a portion of the operational states. Finally, an estimated life of the mechanical device may be determined based on the plurality of operational states experienced by the mechanical device within the predetermined time range. Each of the operating states experienced can correspond respectively to a specific wear and tear, i.e. a reduction of the life span, and thus, e.g., the estimated life of the mechanical device may be adjusted based on these wear and tear.

In an embodiment according to the present disclosure, the method further includes predetermining a maximum range of load values, the maximum range of load values being divided into a plurality of sub-ranges, wherein the particular range of load values includes at least one of the sub-ranges. The maximum load value range may e.g. be an effective operating range of the mechanical device. The maximum temperature range [0° C., 69° C.] may e.g. be given in advance and divided into sub-ranges for every 3° C., i.e. [0° C., 3° C.], [3° C., 6° C.], . . . [66° C., 69° C.]. In this case, the temperature spectrum shown in FIG. 2 may, e.g., be in one of the following sub-ranges [0° C., 3° C.], [3° C., 6° C.], . . . [15° C., 18° C.]. Referring to the table above, these sub-ranges can be combined with lubricant states (lightly, moderately and heavily contaminated) to form the operating state of the mechanical device.

In an embodiment according to the present disclosure, each operational state may also correspond to a corresponding particular range of load values in the plurality of load spectra and a specific degree of contamination in the lubricant state. That is, the operational state of the mechanical device may be determined in multiple dimensions. The plurality of operating states of the mechanical device may be a combination of a first load spectrum, a second load spectrum and a lubricant state. The first load spectrum may e.g. be a temperature spectrum and the second load spectrum may e.g. be a torque spectrum. In this case, the first load spectrum, the second load spectrum and the lubricant state form a three-dimensional coordinate system, and each operational state is a vector in the three-dimensional coordinate system, i.e. corresponding to a particular load range in the first load spectrum, a particular load range in the second load spectrum and a particular degree of contamination in the lubricant state.

FIG. 5 illustrates a flowchart of a method 500 for determining an estimated life of a mechanical device according to another embodiment of the present disclosure. Method 500 has steps S110, S120, and S130 shown in FIG. 1. They are distinguished in that step S140 in method 100 is replaced by the following steps, step S510: determining the corresponding reference life of the mechanical device for each of the plurality of operating states, and step S520: determining an estimated life of the mechanical device based on the reference life corresponding to each of the plurality of operating states. In step S510, according to an embodiment of the present disclosure, a corresponding reference life of the mechanical device may be determined separately for each of the plurality of operating states based on predetermined criteria or methods.

The mechanical device shall have a corresponding reference life for one of the operating states. The reference life may e.g. be given by a predetermined standard for the life of the mechanical device, such as ISO/TS 16281, or determined by other predetermined methods/algorithms. Within a predetermined time range, the mechanical device undergoes a plurality of operating states that correspond to a plurality of reference life. These reference lives are taken into account simultaneously when determining the estimated life of the mechanical device. It is possible e.g. to establish a distribution of these reference lives and to determine the estimated life of the machinery based on statistical characteristics of the distribution of the reference lives, wherein the statistical characteristics may e.g. be the median or the mean, e.g. the harmonic mean, the arithmetic mean and the geometric mean, of the reference lives.

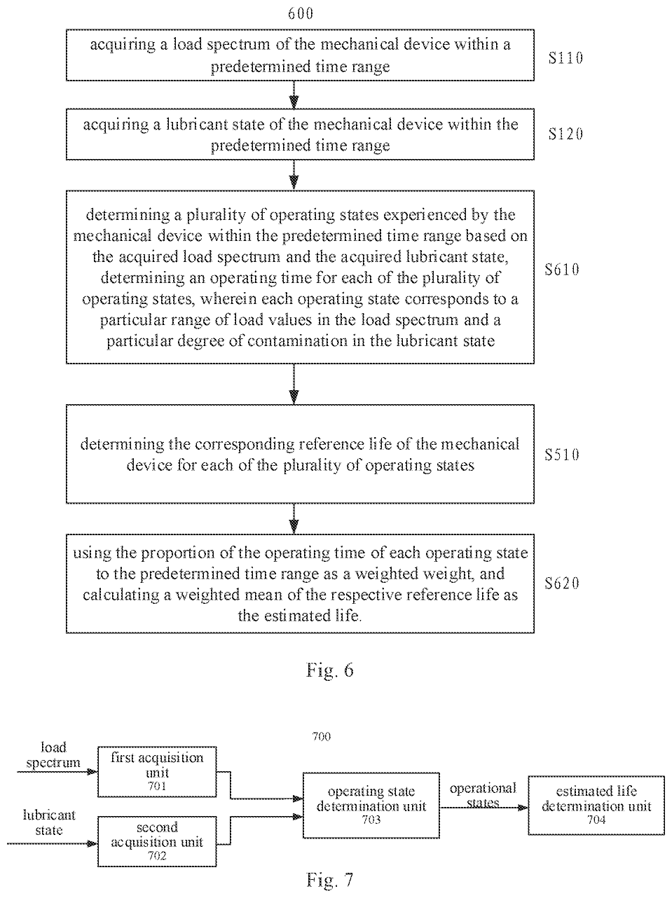

FIG. 6 illustrates a flowchart of a method 600 for determining an estimated life of a mechanical device according to another embodiment of the present disclosure. Method 600 has steps S110 and S120 shown in FIGS. 1 and 5. Replacing step S130, method 600 then performs step S610. In step 610, determining a plurality of operating states experienced by the mechanical device within the predetermined time range may include determining an operating time for each of the plurality of operating states, according to an embodiment of the present disclosure. Subsequently, as in step S510 of method 500, the corresponding reference life of the mechanical device is determined separately for each of the plurality of operating states. Finally, in step S620, the proportion of the operating time of each operating state to the predetermined time range is used as a weighted weight, and a weighted mean of the respective reference life is calculated as the estimated life.

In an embodiment according to the present disclosure, the weighted mean may include a weighted harmonic mean, a weighted arithmetic mean and a weighted geometric mean. A harmonic weighted mean of the respective reference life can be calculated as the estimated life, according to the following equation.

$${L}_{esti - harm} = \frac{{t}_{s u m}}{\frac{{t}_{1}}{{L}_{ref 1}} + \frac{{t}_{2}}{{L}_{ref 2}} + … \frac{{t}_{n}}{{L}_{refn}}} = \frac{1}{\frac{{U}_{1}}{{L}_{ref 1}} + \frac{{U}_{2}}{{L}_{ref 2}} + … \frac{{U}_{n}}{{L}_{refn}}}$$

where Lₑₛₜᵢ₋ₕₐᵣₘ denotes the estimated life of the mechanical device calculated using the weighted harmonic mean; t₁, t₂, . . . tₙ denote the operating time of the mechanical device from operating state 1 to operating state n, respectively, and tₛᵤₘ denotes the total operating time of the mechanical device, i.e., the predetermined time range, tₛᵤₘ=t₁+t₂+ . . . tₙ and U₁, U₂, . . . Uₙ denotes the weighted weight, i.e., the proportion of the operating time from operating state 1 to operating state n to the predetermined time range (total operating time), thus U₁=t₁/tₛᵤₘ·U₂=t₂/tₛᵤₘ, . . . Uₙ=tₙ/tₛᵤₘ, and U₁+U₂+ . . . Uₙ=1; L_{ref1}, L_{ref2}, . . . , L_{refn} denotes the reference life of the mechanical device corresponding to the operating state 1 to the operating state n, respectively.

The weighted arithmetic mean of the respective reference life can be calculated as the estimated life, according to the following equation.

$${L}_{esti - arith} = \frac{{t}_{1} {L}_{ref 1} + {t}_{2} {L}_{ref 2} + … {t}_{n} {L}_{refn}}{{t}_{sum}} = {U}_{1} {L}_{ref 1} + {U}_{2} {L}_{ref 2} + … {U}_{n} {L}_{refn}$$

where Lₑₛₜᵢ₋ₐᵣᵢₜₕ denotes the estimated life of the mechanical device calculated using an weighted arithmetic mean; t₁, t₂, . . . tₙ denotes the operating time of the mechanical device from operating state 1 to operating state n, respectively, and tₛᵤₘ denotes the total operating time of the mechanical device, i.e., the predetermined time range, tₛᵤₘ=t₁+t₂+ . . . tₙ and U₁, U₂, . . . Uₙ denotes the weighted weight, i.e., the proportion of the operating time from operating state 1 to operating state n to the predetermined time range (total operating time), thus U₁=t₁/tₛᵤₘ·U₂=t₂/tₛᵤₘ, . . . Uₙ=tₙ/tₛᵤₘ, and U₁+U₂+ . . . Uₙ=1; L_{ref1}, L_{ref2}, . . . , L_{refn} denotes the reference life of the mechanical device corresponding to the operating state 1 to the operating state n, respectively.

The weighted geometric mean of the respective reference life can be calculated as the estimated life, according to the following equation.

$${L}_{esti - geo} = \sqrt[{t}_{sum}]{{L}_{ref 1}^{{t}_{1}} \cdot {L}_{ref 2}^{{t}_{2}} \cdot … {L}_{refn}^{{t}_{n}}}$$

where L_{esti-geo} denotes the estimated life of the mechanical device calculated using the weighted geometric mean; t₁, t₂, . . . tₙ denote the operating time of the mechanical device from operating state 1 to operating state n, respectively, and tₛᵤₘ denotes the total operating time of the mechanical device, i.e., the predetermined time range, tₛᵤₘ=t₁+t₂+ . . . tₙ; b_{ref1}, L_{ref2}, . . . , L_{refn} denotes the reference life of the mechanical device corresponding to the operating state 1 to the operating state n, respectively.

The present disclosure also provides a method for determining a remaining useful life (RUL) of a mechanical device. The estimated life of the mechanical device can first be determined according to the method described in detail above. Then, the time for which the mechanical device has been used can be determined, e.g. starting from a time point when the mechanical device was put into use and ending at a current time point. Finally, the remaining useful life of the mechanical device can be determined based on the estimated life of the mechanical device and the time already in use, e.g. the remaining useful life of the mechanical device is equal to the estimated life of the mechanical device minus the time already in use, i.e.

$${L}_{rul} = {L}_{esti} - {L}_{used}$$

wherein Lᵣᵤₗ denotes the remaining useful life of the mechanical device, and Lₑₛₜᵢ denotes the estimated life of the mechanical device, and L_{used} denotes the time the mechanical device has been in use.

The weighted means listed are examples only, and other existing or possible future weighted means should be included here by reference.

The mechanical device may be, e.g., a wind turbine, and in particular may be a main shaft of a wind turbine, which is in a harsh outdoor environment, where its ambient temperature fluctuates over a wide range and where its lubricant state progressively deteriorates with non-stop operation. In determining the estimated life or remaining useful life of the mechanical device, the method for determining the estimated life of the mechanical device and the method for determining the remaining useful life of the mechanical device according to the present disclosure can take into account all the operating states, i.e., combinations of different load conditions and different lubricant states, that the mechanical device has experienced from the time it was put into operation until the current time point. Since different operating states have different effects on the life of the mechanical device, the methods according to the present disclosure are capable of accurately determining or adjusting the estimated life of the mechanical device based on the various operating states experienced by the mechanical device. In addition, the number and duration of the various operating states experienced by the mechanical device can change over time. The methods according to the present disclosure are able to take such changes into account and are therefore able to determine the life and remaining useful life of the mechanical device in real time.

FIG. 7 illustrates a device 700 for determining an estimated life of a mechanical device according to an embodiment of the present disclosure, the device 700 includes: a first acquisition unit 701 for acquiring a load spectrum of the mechanical device within a predetermined time range; a second acquisition unit 702 for acquiring a lubricant state of the mechanical device within the predetermined time range; and an operating state determination unit 703 that is connected to the first acquisition unit 701 and second acquisition unit 702 and acquires the load spectrum and the lubricant state, and determines a plurality of operating states experienced by the mechanical device within the predetermined time range based on the load spectrum and the lubricant state, wherein each operating state corresponds to a particular range of load values in the load spectrum and a particular degree of contamination; and an estimated life determination unit 704 that is coupled to the operational state determination unit 703 for determining an estimated life of the mechanical device based on the plurality of operational states of the mechanical device within the predetermined time range.

In an embodiment according to the present disclosure, the first acquisition unit 701 may e.g. be a sensor arranged inside the mechanical device, and different sensors may be employed for different types of loads. E.g., for temperature, a temperature sensor may be used, for torque, a torque sensor may be used, for rotational speed, a rotational speed sensor may be used, and for radial and/or axial force a force sensor may be used. The second acquisition unit 702 may e.g. also be a sensor arranged inside the mechanical device to detect a lubricant state in the mechanical device in real time. Alternatively, the second acquisition unit 702 may also acquire the lubricant state only periodically, e.g. every few days or months. As shown in FIG. 4. In this case, the device 700 may determine an estimated life of the mechanical device based on the previously captured lubricant state at the intervals.

In addition, according to an embodiment of the present disclosure, the estimated life determination unit 704 in the device 700 may also subtract the time that the mechanical device has been in use from the estimated life, thereby acquiring the remaining useful life of the mechanical device.

The block diagrams of the circuits, units, devices, apparatuses, equipment, and systems involved in this disclosure are intended to be exemplary only and do not purport to require or imply that they must be connected, arranged, or configured in the manner illustrated in the block diagrams. As those skilled in the art will recognize, the circuits, units, devices, apparatuses, equipment, and systems may be connected, arranged, and configured in any manner as long as they are capable of achieving the desired purpose. The circuits, units, devices, and appliances involved in this disclosure may be implemented in any suitable manner, such as by using special purpose integrated circuits, field programmable gate arrays (FPGAs), and the like, or by using general purpose processing units in combination with programs.

It should be understood by those skilled in the art that the above specific embodiments are only examples and not limitations, and various modifications, combinations, partial combinations, and substitutions of the embodiments of the present disclosure may be made according to design needs and other factors, as long as they are within the scope of the appended claims or their equivalents, which fall within the scope of the rights that the present disclosure is intended to protect.

What is claimed is:

1. A method for determining an estimated life of a mechanical device, the method comprising:
acquiring a load spectrum of the mechanical device within a predetermined time range;
acquiring a lubricant state of the mechanical device within the predetermined time range;
determining a plurality of operating states experienced by the mechanical device within the predetermined time range based on the acquired load spectrum and the acquired lubricant state, wherein each operating state corresponds to a particular range of load values in the load spectrum and a particular degree of contamination in the lubricant state; and
determining an estimated life of the mechanical device based on the plurality of operating states of the mechanical device within the predetermined time range.

2. The method according to claim 1, wherein determining an estimated life of the mechanical device based on the plurality of operating states of the mechanical device within the predetermined time range comprises:
determining the corresponding reference life of the mechanical device for each of the plurality of operating states, and
determining an estimated life of the mechanical device based on the reference life corresponding to each of the plurality of operating states.

3. The method according to claim 2, wherein determining the corresponding reference life of the mechanical device for each of the plurality of operating states comprises: determining the corresponding reference life of the mechanical device for each of the plurality of operating states according to a predetermined criterion or methodology regarding the life of the mechanical device.

4. The method according to claim 2, wherein determining a plurality of operating states experienced by the mechanical device within the predetermined time range comprises:
determining an operating time for each of the plurality of operating states;
wherein determining an estimated life of the mechanical device based on the reference life corresponding to each of the plurality of operating states comprises:
using the proportion of the operating time of each operating state to the predetermined time range as a weighted weight, and calculating a weighted mean of the respective reference life as the estimated life.

5. The method according to claim 4, wherein the weighted mean comprises: a weighted harmonic mean, a weighted arithmetic mean, and a weighted geometric mean.

6. The method according to claim 1, wherein acquiring a lubricant state of the mechanical device within the predetermined time range comprises:
acquiring a discrete lubricant state of the mechanical device within the predetermined time range, and
deriving a continuous lubricant state within the predetermined time range based on the discrete lubricant state and a time point at which the lubricant is cleaned within the predetermined time range.

7. The method according to claim 1, wherein the particular degree of contamination comprises: lightly contaminated, moderately contaminated and heavily contaminated.

8. The method according to claim 1, further comprises predetermining a maximum range of load values, the maximum range of load values being divided into a plurality of sub-ranges, wherein the particular range of load values comprises at least one of the sub-ranges.

9. A method for determining the remaining useful life of a mechanical device, comprising:
determining an estimated life of the mechanical device using the method of claim 1, and
determining the time the mechanical device has been in use, and
determining the remaining useful life of the mechanical device based on the estimated life of the mechanical device and the time the mechanical device has been in use.

10. A method for determining the remaining useful life of a mechanical device, comprising:
determining an estimated life of the mechanical device using the method of claim 5, and
determining the time the mechanical device has been in use, and
determining the remaining useful life of the mechanical device based on the estimated life of the mechanical device and the time the mechanical device has been in use.

11. An equipment for determining an estimated life of a mechanical device, the equipment comprising
a first acquisition unit for acquiring a load spectrum of the mechanical device within a predetermined time range;
a second acquisition unit for acquiring a lubricant state of the mechanical device within the predetermined time range;
an operating state determination unit that is connected to the first acquisition unit and the second acquisition unit and acquires the load spectrum and the lubricant state and, determines a plurality of operating states experienced by the mechanical device within the predetermined time range based on the load spectrum and the lubricant state, wherein each operating state corresponds to a particular range of load values in the load spectrum and a particular degree of contamination in the lubricant state; and
an estimated life determination unit that is connected to the operating state determination unit, determines an estimated life of the mechanical device based on the plurality of operating states of the mechanical device within the predetermined time range.
